(19) United States
(12) Patent Application Publication
(10) Pub. No.: US 2016/0272943 A1
(43) Pub. Date: Sep. 22, 2016

(54) Apparatus and method for closed system recovery of cells from tissue samples

(71) Applicant: InGeneron Incorporated, Houston, TX (US)

(72) Inventors: Greg Stone, Houston, TX (US); Ron Stubbers, Houston, TX (US); Michael E. Coleman, The Woodlands, TX (US); Eckhard Alt, Houston, TX (US)

(21) Appl. No.: 14/757,308

(22) Filed: Dec. 15, 2015

Related U.S. Application Data
(63) Continuation-in-part of application No. 13/998,079, filed on Sep. 27, 2013.
Application No. 13/998,079 is a division of application No. 13/329,143, filed on Dec. 16, 2011, now Pat. No. 8,951,513.

(51) Int. Cl. C12N 5/0775; C12M 1/26; C12M 1/00

(52) U.S. Cl. CPC C12N 5/0667; C12M 47/02; C12M 47/12; C12M 33/10; C12N 2509/00

(57) ABSTRACT

A closed system apparatus and method for isolation and recovery of desired cells from body tissue includes a first container for tissue dissociation by subjection to centrifugation over one or more cycles of rotational acceleration and deceleration, a particle filter to filter out non-dissociated tissue while passing desired cells to a second container for separating the desired cells from connective tissue, unwanted material, liquid, and dissociation-aiding reagent(s) by subjection of the second container to combined centrifugal and gravitational forces that also causes concentration and sedimentation of the cells into a pellet for removal through a valve in the bottom of the second container.

### CROSS-REFERENCE TO RELATED APPLICATIONS

This application claims priority of and is a continuation-in-part of co-pending nonprovisional application Ser. No. 14/757,229, filed 7 Dec. 2015, now abandoned. It is also related to co-pending nonprovisional application Ser. No. 13/998,079, filed 27 Sep. 2013, which is a division of U.S. Pat. No. 8,951,513 (“the '513 patent”), issued 10 Feb. 2015, originally filed as nonprovisional application Ser. No. 13/329,143 on 16 Dec. 2011. The disclosure of those and all other patents and patent applications cited in this specification are hereby incorporated by reference in their entirety in the disclosure of this application.

### FIELD OF THE INVENTION

This invention relates generally to closed system methods and apparatus for recovery of cells, principally regenerative cells, from body tissue obtained from human or other mammalian donor subjects, and more particularly to recovery of cells from tissue samples by means of a two-tube recovery system together with intervening cycles of centrifugation.

### BACKGROUND

Current strategies in regenerative medicine aim towards replacing tissue that undergoes an increased apoptosis rate. That means that within the organ there is a net loss of functional cells because more cells are dying than are being replaced. Therefore, the transfer of regenerative cells, stem cells, progenitor cells, and other types of reparative cells (collectively referred to herein as “regenerative cells”) from one location to the site of renewal is a therapeutic approach to restore the organ back to an equilibrium. Research at our laboratories has shown that regenerative cells are present in tissues of the mammalian body, indeed in every organ, primarily located in the vascular and perivascular space, such as in the vessel wall attached to the lamina elastica interna. One population of these cells is able to replace the stroma of an organ, the other part of these cells is capable to differentiate into the respective parenchym of the specific organ. Each organ can be compared to a house, where the stroma made from fibroblasts and consisting of extracellular matrix can be compared to walls of bricks and mortar in a house, the piping in a house corresponds to blood vessels of the organ and nerves represent the electrical wiring in the walls. Inside these houses, in each organ one has a certain type of inhabitants, such as liver cells, heart cells, bone cells, cartilage or fat cells, known as the parenchym of an organ.

In order to restore function to a dysfunctional organ, it is important to provide both the stroma, meaning the housing that forms the walls of the organ, and the inhabitants, meaning the specific parenchymal cells.

Techniques for isolation of regenerative cell populations and their uses abound, such cells including pre-adipocytes, fibroblasts, pluripotent stem cells, endothelial cells, endothelial progenitor cells, and other supporting cell types. Isolated, or purified cell populations, have been shown to have various potential therapeutic applications. Pre-adipocytes may provide a durable filler for wrinkles or other cosmetic skin defects; fibroblasts may have utility to treat wrinkles and skin wounds; endothelial cells and endothelial progenitor cells may contribute to neovascularization supplying oxygenated blood to ischemic tissue, as well as contributing factors that may be protective to damaged tissues; and pluripotent stem cells may have the capacity to treat a number of conditions, because of the ability of such cells to differentiate into various cell types and tissues.

The use of cell preparations for therapeutic application, such as tissue repair, may be complicated by the presence of proteins, by-products of the isolation process, and other cell types depending on the medical application. For example, the presence of leukocytes may cause immune system inflammatory problems in certain indications. This could be life threatening when the cell preparation is used in tissue repair in the heart, for example.

In light of the foregoing, it would be beneficial to develop an efficient and cost effective system and method for isolating and purifying cell populations with good yields in clinical settings, which require isolation in a relatively rapid time frame. Such a system and method would benefit from wide applicability to address the need for regenerative cells. The introduction and acceptance of such new technologies into clinical practice is dependent on their cost effectiveness, safety, and ease of use.

A method and means to recover regenerative cells capable of restoring organ function is to dissociate them from subcutaneous fat tissue, i.e., adipose tissue, because that tissue is rich in blood vessels and is not essential for life. Most people are capable, even quite willing, to donate several grams of adipose tissue from their bodies.

Regenerative cells have properties beyond repair or replacement of damaged tissue, including capability to reduce inflammation, to generate new blood vessels, and to construct new tissue. Recovery of such cells may be sought to put them to immediate use, or to store them for later use, through banking, cryopreservation, or other known means. Although regenerative cells are available in all soft tissues, they may not be present in a particular tissue or tissues in quantities desired for a particular use or intervention. It is desirable, therefore, in such instances to collect regenerative cells from tissue locations where they are currently present in relatively large quantity but unneeded, and/or to concentrate them, and thereafter apply them to tissue areas in need of repair, protection, or construction.

Autologous grafting of tissue harvested by lipoaspiration is a common procedure in cosmetic surgery for both small (e.g., nasolabial folds) and large (buttocks or breast) volume filling applications. The primary benefits of this procedure termed “autologous fat grafting” are lower cost versus synthetic fillers and no immune rejection since the patient's own tissue is used. Currently, multiple methods of lipoaspirate collection and processing are employed to obtain tissue for grafting. Factors that determine clinical outcomes following autologous fat grafting have not been fully elucidated. However, it is widely recognized that improving the persistence of the graft is an area of significant need.

Where regenerative cells are sought to be acquired from body tissue other than adipose tissue of a donor subject, it is generally found that the quantity to be obtained from such other tissue is insufficient for an intended immediate use. Such situations would require that the cell population be expanded, necessitating a delay, of perhaps weeks, before a sufficient population of regenerative cells may obtained for use. And population expansion requires that the acquired cells be cultured, which modifies the surface protein characteristics of the cells. And in the case of recovery from adipose tissue, systems, devices, and processes heretofore proposed to acquire a sufficient population of regenerative cells therefrom are overly cumbersome and/or expensive for extensive use.

It would be highly desirable to provide closed system apparatus and methods for relatively simple, convenient, efficient and inexpensive processing that enables extraction and concentration of a concentrated population of regenerative cells from adipose tissue. It would also be desirable to do so in a sufficiently short period of time for the recovered cells to be implanted in the same surgical procedure in which the tissue itself was obtained.

### SUMMARY OF THE INVENTION

To that end, the present invention is directed to a system and method for processing of adipose tissue or lipoaspirate for isolation and concentration of regenerative cells therefrom. The system or apparatus of the invention includes a set of disposable items for processing the tissue and dissociating enzyme, and a device for agitating and heating some of the disposable items in which the tissue is processed.

The present invention provides several important advantages over existing technology. First, it provides a simple method to efficiently separate the non-adipocyte cell fraction from the significant fraction of lipid-filled adipocytes, oil, enzymatic and other protein processing by-products, which are present in a cell mixture that results from disaggregated adipose tissue or lipoaspirate. This improvement reduces complexity and capital equipment requirements. Second, the present invention offers the end user a familiar form factor, utilizing syringes and luer connections easily integrated with typical clinical use. Third, the system of the present invention can be produced economically in a practical and cost-efficient embodiment, to enable substantial reduction of per-treatment costs.

The present invention is especially effective in rapid, efficient, and cost-effective isolation of desirable cell populations for use as autografts in target tissues, although numerous other applications, including but not limited to those mentioned earlier herein, will become apparent to the reader of this specification. As such, a multitude of tissues can be processed by this system in order to recover target cells from the respective tissue such as heart, liver, bone or brain, including tumors of the human or animal body.

In one aspect, the invention resides in an apparatus for isolation and purification of a cell mixture from a multitude of tissues, the apparatus including a closed and vented tissue processing vessel, filter for retention of unprocessed tissue, and a closed and vented concentration and purification vessel with reservoir and an opening on the top for filling with pre-processed tissue and a second opening on the bottom for extraction of the purified cell mixture. The separation between for example a regenerative cell mixture and the unwanted remnants of the enzyme enabled separation process is performed by gravity-related centrifugation. In that way, after centrifugation a separation between the regenerative cells with higher specific gravity and the relatively lower specific gravity of lighter cells, debris, oil, adipose cells, collagen particles and un-wanted remaining enzyme occurs. In a particular embodiment, the apparatus includes a closed tissue processing vessel preferably tubular in shape where one end of the tubular shape is essentially conical in shape, and the other end includes a sterilizing filtered vent (preferably, of 3 micron pore size) for pressure equalization, and a connection based on the luer standard.

According to a presently preferred but exemplary embodiment, a reusable Tissue Processing Unit (TPU) and a set of inexpensive closed sterile disposable containers are employed for processing of adipose tissue or lipoaspirate to isolate and remove a regenerative cell population from the body tissue, and to recover the regenerative cells of the mixture in concentrated (i.e., pellet form) for subsequent use. The TPU performs functions including repetitive acceleration and deceleration, swing out centrifugation, with a programmable capability to repetitively ramp up to a selected speed, hold that speed, and quickly ramp back down to a stop. The TPU includes a heated chamber and a rotor with buckets in which containers are to be placed for subjection to spinning. Feedback from the TPU gives the user information regarding current function selected from among specific functions, including start and stop data.

Among the set of closed sterile disposable components are assemblies including a processing tube (also referred to herein as a container), a particle filter, and a wash tube (also referred to herein as a container). In the exemplary embodiment, the processing tube may be substantially cylindrical in form, composed of a material that allows viewing of the amount and status of processing of the body tissue within the tube. The flat end of the cylinder has a closable female luer port for receiving the body tissue to be processed, and a closed sterile filtered vent for pressure equalization. The opposite end of the processing tube is preferably of conical shape, to conform to the shape of the bucket in which it will be seated. After the body tissue to be processed has been inserted within the processing tube, together with an enzyme and/or other substances to aid dissociation, the tube is placed in a bucket on the rotor in the chamber of the TPU. The rotor with its inverted buckets, wherein the end of each bucket closer to the rotor axis lies below the opposite end of the bucket, is then repetitively accelerated and decelerated to subject the tissue/enzyme mixture to a selected regimen of rotational acceleration and deceleration, ultimately for agitation to aid in enhanced dissociation of cells from the tissue in the tube. Concurrently, the chamber is heated to a selected controlled temperature, preferably at or about 37° C., to further aid enzyme-induced dissociation but still maintain cell viability.

In the preferred embodiment, the processing tube is secured in a respective bucket fixedly attached to the rotor of the centrifuge. The conical end of the tube is firmly seated in the correspondingly shaped taper of the receptacle of the bucket, which serves to allow a maximum volume of material to be processed in the processing tube. The bucket initially is in a standard swing out position, so it can swing out from a vertical position with increasing centrifugal force to a more horizontal orientation.

In addition to this swing out mode, the bucket can be lifted and fixed on the rotor (or a rotor arm), whereby the conical end of the processing tube seated therein is positioned away from (i.e, distal to) the rotor's central axis, and the orientation of the bucket is such that the tube therein is angled slightly upwardly, for example by 10 to 30 degrees, to elevate the conical end relative to (i.e., higher than) the flat cylinder end. With this positioning, activated by a fixing mechanism of the rotor, material within the processing tube at rest is urged by gravity toward the rotor central axis, and away from the conical end. And when the TPU rotor is spun, centrifugal force on the contents of the processing tube causes them to move away from the flat end (and the rotor axis) and toward the conical end of the tube in a circular manner. Thus, repetitive and cyclical acceleration and deceleration of the rotor has the effect of agitation of the processing tube contents to speed dissociation of tissue, aided by the enzyme, toward separating the cells from the tissue matrix. By virtue of the shape of the tube conforming to the shape of the bucket in which it is held in the centrifuge, a maximum volume of material can be processed in the processing tube. Alternatively, if the bucket is rotatably attached (i.e. in a swing out mode) to the rotor (or rotor arm), the conical end of the processing tube is oriented downwardly and vertically at rest, but swings upwardly, horizontally and outwardly away from (i.e, distal to) the rotor's central axis in response to spinning of the rotor, but never to more than 90 degrees. This results in heavier material within the tube moving toward the conical end by gravitational forces, and lighter materials with lower specific gravity remaining largely at the flat end.

The relatively flat particle filter of the exemplary embodiment has a male luer port on one end and female luer port on its other end. A mesh occupies the interior of the particle filter in intervening relation between the two ends, with spaces or openings in the mesh sized (e.g., in a range of preferably 40 microns (micrometers, or μm) to 300 μm) to allow passage of cells of interest therethrough, while pieces of non-dissociated tissue as well as any other particles relatively larger than the size of the openings are trapped by the mesh. For use in processing the body tissue, the male luer end of the filter is connected to the female luer at the flat end of the processing tube. A syringe with its plunger fully extended into the syringe barrel is connected to the female luer end of the filter. Thereby, when the syringe plunger is withdrawn along the barrel by pulling the syringe handle outwardly, the cellular/enzyme/particle mixture that resulted from the agitation/heating and filtration steps or stages of the process is vacuum-extracted from the processing tube. That is, the mixture is transferred from the processing tube through the filter, where the larger particles are trapped in the filter mesh, and the remaining cell/enzyme mixture is drawn into the syringe.

The regenerative cells released by the enzyme in the mixture from the adipose tissue are then separated from the unwanted tissue components, and undergo volume-reduction, by use of the wash tube. In the exemplary embodiment, the wash tube is cylindrical with a conical taper on its bottom end that opens into a cylindrical reservoir of reduced diameter relative to the largest diameter of the wash tube. In contrast to the processing tube, the wash tube has two openings in addition to the vented port. One of these openings is at the flat top end of the wash tube, and the second at the conical end. This second opening is initially closed by a reduced-length-stem cap on a luer port with a swabable silicone insert. The syringe now containing the dissociated cell mixture is then connected at its male luer end to a female luer on the top flat circular end of the closed wash tube, to enable the operator to transfer material from the barrel of syringe by depressing the syringe plunger into the barrel to discharge its contents, thereby loading them into the wash tube.

Enzyme reagent, excess liquid, and other material with lower specific gravity may then be separated from the higher specific gravity cell population of the mixture. This is accomplished in part by securely seating the conical end of the loaded wash tube in the tapered receptacle of a rotor bucket of the TPU, with the flat end of the tube proximate the rotor, and the alignment of the wash tube corresponding to that of the earlier-described placement of the processing tube in a rotor bucket. Spinning the rotor subjects the contents of the wash tube to centrifugal force that moves the contents toward the conical (and reservoir) end of the tube distal from the rotor axis. The centrifugation is performed at a speed sufficient to cause sedimentation of the cell mixture in the bottom (conical end) of the wash tube, and into the reservoir as it forms a concentrated regenerative cell pellet, while unwanted cell debris remains above following its lower specific gravity.

The pellet may then be removed from the wash tube by application of a second syringe to the port at the reservoir end of the bottom of the wash tube. In the exemplary embodiment, that end of the reservoir is a membrane or silicone cup that is pierced by the needle of a syringe to allow withdrawal of the cell pellet from the reservoir, but that retains the pellet in the reservoir when undisturbed. Alternatively, the reservoir end may have a female luer port with a normally closed membrane adapted to be opened by penetration of a male luer connector on a syringe, acting as a valve that during processing and centrifugation is closed, but can be opened at the end of a procedure to remove material with higher specific density that locates at the bottom with gravitational force. This type of luer port is referred to in the field as a ‘swabable’ female luer port as it may be swabbed with alcohol for disinfection.

Accordingly, in its simplest configuration, an embodiment of the present invention comprises at least a first closed sterile container for dissociation of cells of interest from body tissue (such as adipose tissue, other organs of the body or even from tumors and bone), a second closed sterile container for separation of the isolated and recovered cells from debris and other cells, connective tissue and matrix material, wash liquid, and any substance used to aid dissociation, and concentration of the isolated and recovered cells into a pellet. The cell pellet (contained within a small volume of 1 mL to 5 mL, preferably 2 mL) forms at the bottom of the wash tube after centrifugation, and can be removed with a syringe from the bottom opening. A sterile 0.2 μm to 0.5 μm filter is also part of this embodiment, and a vent of a container compensates and equilibrates the internal pressure change while filling or removing material inside the containers.

More specifically, a presently preferred but exemplary embodiment comprises a TPU for isolation and recovery of regenerative cells from body tissue, preferably adipose tissue or lipoaspirate, the TPU including a chamber with rotor and rotor bucket(s) for receiving a first sterile container with closable ports for containment of the body tissue and enzyme therein to be subjected to repetitive acceleration and deceleration and heating to aid dissociation of a cell mixture from tissue by use of an enzyme mixture; a particle filter for transferring material from the first to the second container by passing the cell and enzyme mixture while precluding passage therethrough of tissue and other substances larger than the size of mesh openings within the filter; and a second closed sterile container for receiving the filtered material comprising the cells, especially regenerative cells, enzyme, and connective material that passed through the mesh, together with wash liquid from a separate source, for subjection to a simple centrifugation run to isolate and recover regenerative cells by sedimentation and concentration into pellet form segregated from other material in the second container; and an integral reservoir of the second container for receiving the pellet, to enable its removal for a prescribed medical procedure.

A first closed sterile container of the apparatus is preferably cylindrical, tapered along a portion of its length to form a cone-shape at one of its ends, and preferably includes a pair of ports disposed on its other flat circular end. Of these, a first closable port is provided for ingress of the body tissue and enzymes used to aid dissociation, and for egress of dissociated tissue and cell mixture. The first closable port is preferably a tapered luer port. A second opening has a pressure equilibration filter for its closure, a vent for equilibration of pressure while filling, preferably with a sterile filter membrane of 0.2-0.5 μm that allows air, but not liquid or particles such as bacteria, to flow through it.

A second closed sterile container of the apparatus is also preferably cylindrical, with a flat circular end and a tapered conical opposite end having a luer connection port on both ends. A first closable port on the container's flat end enables entry of filtered material including cells, enzyme and connective material. The luer connection port at the opposite tapered end of the container is associated with a reservoir, which is itself is closed by a membrane or swabable flexible seal that is penetrable by a tapered male luer tip of a syringe. The flexible seal may be a silicone cup having a slit therein that is normally closed but opened when the syringe tip is inserted therein.

The invention also encompasses a method for regenerative cell recovery from body tissue, the method preferably comprising placing the tissue to be dissociated into a first closed sterile container along with enzyme reagent, heating and agitating the tissue to promote dissociation of the tissue in the container, transferring the now dissociated tissue from the container through a particle filter for removal of materials larger than the cells to be recovered, and the then-remaining cell and enzyme mixture from the filter into a second closed sterile container, subjecting the cell and enzyme mixture to at least one wash cycle with wash fluid and a run of centrifugation to concentrate the cell population from the cell portion of the mixture by sedimentation thereof, followed by retention as a cell pellet in a reservoir disposed at an end of the second container, and extracting the resulting cell pellet from the reservoir using a syringe to open a flexible retainer of the reservoir.

In one aspect of the invention, the dissociation reagent preferably comprises a proteolytic enzyme of one or more of a protease and collagenase. When filled into the first container, the volume of the fill leaves sufficient air space at the top of the container to enhance agitation of the tissue and reagent. This is performed by placing the first container in the TPU, which is adapted or programmable to perform repeated automated cycles of acceleration and deceleration of that container after placement therein. The end of the container distal from the axis of rotation of centrifugation is positioned at a level higher than the other end, which is proximal to the axis of rotation. Thereby, the centrifugal forces of acceleration move the combination of tissue and dissociation reagent upward within the container and outward relative to the axis of rotation, and when the deceleration commences toward cessation, the oppositely directed gravitational force moves the combination downward in the container and toward the axis of rotation in a rotational movement.

Preferably, a sterile luer-end syringe is used to transfer the dissociated tissue from the first container, first through the filter and then into the second container. More specifically, movement of the dissociated tissue from the first container through the particle filter and into the second container is accomplished utilizing a configuration in which the first container has a female luer port coupled by an operator (i.e., manually) to a male luer port of the filter, and a female luer port of the filter is coupled by the operator to the luer connection at the tip of the syringe, such that withdrawing the fully-entered plunger of the syringe from its barrel via the syringe handle vacuum-extracts (i.e., draws) the cell mixture into the barrel. The syringe is thereby fully loaded with filtered cell/enzyme mixture for transfer via its male luer connector into the second container via the latter's female luer port. The method further includes washing and centrifuging the mixture in the second container to cause the cells of interest to move to the cone-shaped bottom of the container for concentration by sedimentation thereof to form the cell pellet. The other portions of the mixture including enzymes and other substances of lower specific gravity remain suspended in the second container for subsequent removal and discarding as waste. The purified regenerative cell pellet comprising a concentrated cell population is extracted from the reservoir at its bottom for subsequent washing steps and use in any of the medical procedures addressed earlier herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the systems, apparatuses and methods described in the present application, including definitions, shall be controlling. The cited examples are illustrative only and not intended to be limiting.

Other features, aspects, objects and attendant advantages of the invention will become apparent from the following drawings and detailed description.

### BRIEF DESCRIPTION OF THE DRAWINGS

FIG. 1 is a simplified flow chart illustrating the steps or stages of processing a donor's body tissue to obtain a concentrated regenerative cell population therefrom, which may be implanted in the same procedure in a recipient subject (who may be the donor) or may be banked for storage in contemplation of subsequent implantation in a recipient;

FIG. 2 is a transparent perspective view of a centrifuge and chamber of a TPU for dissociation, separation and recovery of cells from a body tissue sample according to an embodiment of the invention;

FIG. 3 is a set of stylized side views of functional disposable items of apparatus used in a presently preferred apparatus and method of the invention, in which FIG. 3A represents a processing tube, FIG. 3B represents a particle filter, FIG. 3C represents a wash tube, and FIG. 3D represents a syringe;

FIG. 4 is a simplified cross-sectional side view of the placement and positioning of tissue and other containers in the centrifuge chamber of FIG. 2;

FIG. 5 is a partial perspective view of a centrifuge rotor with buckets or mounts pivotally fastened thereto but arranged for fixed orientation in a modified centrifuge of a TPU for the apparatus and method of the invention;

FIG. 6 is a partial phantom perspective views of a centrifuge rotor with a bucket or mount pivotally fastened thereto for pivotally-determined orientation in the modified centrifuge of FIG. 5; and

FIG. 7 depicts an exemplary closed tissue processing system and flow diagram of an exemplary method of the invention.

### DETAILED DESCRIPTION OF A PREFERRED METHOD AND APPARATUS

The present invention provides provide cost effective means for recovery of cells including a large population of regenerative cells, which include stem cells and early mesenchymal cells plus the whole range of progenitor cells, preferably from their location in subcutaneous adipose tissue, for reasons noted above. The method includes subjecting the removed body tissue (which terminology includes adipose tissue and lipoaspirate, as well as other potential tissue sources of cells, such as various organs, bones and even tumors) to enzymatic and mechanical processes using techniques of treating the tissue, organs and even as yell tumors, all of which contain cells, with enzymes, filtration and sedimentation combined with subjection to controlled heating within the chamber of a centrifuge with a rotor (that may be reconfigured) and one or more cycles of spinning and stoppage of tubular containers of the body tissue in buckets of the rotor, in various stages of processing to recover a purified and concentrated regenerative cell population in a relatively simple and cost-effective manner.

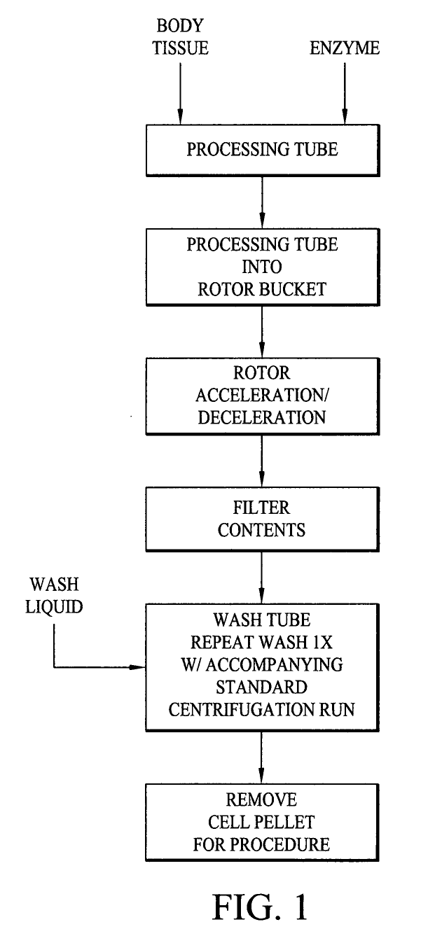

Referring to the process flow chart of FIG. 1, the body tissue to be processed is inserted within a processing tube (the first container, to be described more fully presently), together with one or more dissociation reagents including enzymes, such as proteolytics that may include collagenase and/or neutral protease, and/or other substances to aid dissociation of the tissue. The processing tube is then placed in a bucket on the rotor in the centrifuge chamber of the TPU, and the angular orientation of the bucket and tube is fixed to an upward position. The rotor is then spun to subject the tissue/enzyme mixture to a selected regimen (according to a desired program), preferably at least two cycles of rotational acceleration and deceleration to a stop, the second cycle start substantially immediately after the first cycle ends. The effect of this stage of the process is to repetitively agitate the contents of the processing tube sufficiently to aid in dissociation of cells and other particles from the tissue in the tube. During the cycles, the centrifuge chamber is heated to a selected controlled temperature, preferably at or about 37° C., to enhance the enzymatic dissociation of the tissue but still maintain cell viability.

Following the agitation stage, the dissociated tissue/cellular mixture is subjected to filtration by passage through a particle filter that allows passage of regenerative cells and similarly small particles, but prevents passage of larger pieces of non-dissociated tissue as well as any remaining particles larger than the size of the cells interest. The filtered cells and other particles are vacuum-extracted by withdrawal of the plunger of a syringe coupled through a connector to the output of the particle filter.

The filter stage of the process is followed by a concentration and cleansing stage in which the cell population in the mixture is transferred by the syringe from the filter stage to a cylindrical wash tube (the second container, to be described in detail presently) for further processing. This wash tube, which now contains the cell/enzyme mixture derived from the filter, is placed in the same rotor bucket in swing out mode (along with other wash tubes containing similar mixtures, or filled with dissimilar mixtures for ballast, placed in other rotor buckets) of the centrifuge chamber. A standard centrifugation sedimentation run is performed at a speed sufficient to cause sedimentation of the cells from the enzymes and other remaining substances of lower specific gravity. The resulting concentration of cell population in the form of a cell pellet is captured in a reservoir of the wash tube, from which the pellet is extracted and re-introduced into the wash tube with wash liquid, where another centrifugation run serves to cleanse the concentrated cell pellet. The pellet is again captured in and extracted from the reservoir for use in an applicable medical procedure of any of the types mentioned earlier herein.

The methods and apparatus of the invention are aimed at recovery of regenerative cells from mammalian tissues, including human tissue and animal tissue such as canine, feline, equine, bovine, ovine, or porcine tissues. The invention is particularly useful for recovery of regenerative cells from adipose tissue obtained, for example, by surgical removal or by liposuction (i.e., lipoaspirate), including suction assisted, water jet assisted, or ultrasound assisted liposuction, and combinations thereof.

The methods and apparatus of the invention may be used on site to prepare cellular compositions for administration to a subject, initially by recovery of regenerative cells from the subject as a donor, the cells then prepared as described herein for administration, and followed by administering (e.g., injecting or surgically implanting) the preparation back to the subject from which the cells were recovered (i.e., autologous administration). The cells may be loaded into a delivery device such as a syringe, for injection into the recipient by, for example, subcutaneous, intravenous, intramuscular, or intraperitoneal techniques. For example, the regenerative cells can be injected into blood vessels for systemic or local delivery, into tissue (e.g., cardiac muscle or skeletal muscle), into the dermis (subcutaneous), into tissue space (e.g., pericardium or peritoneum), or other location. Injection of the regenerative platform (i.e., the regenerative cells after preparation for administration) may be performed to augment, repair, reduce inflammation of, reduce pain of, or a combination to, selected tissue in an area proximate the injection site. In some embodiments, one or more additives are added to the cells before administration. For example, the cells can be mixed with other cells, biologically active compounds, biologically inert compounds, demineralized bone, a matrix or other resorbable scaffold, one or more growth factors, or other additive that can enhance the delivery, efficacy, tolerability, or function of the cell population.

The invention also may be used to prepare cellular compositions for growth studies, gene expression studies, differentiation studies, or other research purposes. In addition, the recovered regenerative cell populations in concentrated form may be stored by banking them, such as by cryopreservation with an appropriate medium. In the exemplary embodiment, the processing tube may be a substantially cylindrical clear or translucent container to allow the amount and state of body tissue within the tube and the status of its processing to be viewed by the attending physician, surgeon or other observer.

In brief, the TPU of the apparatus comprises a centrifuge, which has the added capabilities of programmed and controlled heating of its chamber and thereby, of the items being processed; and also of being programmable to rapidly ramp up to a selected rotational speed (in RPM's), hold that speed, and ramp back down to a stop. The centrifuge of the TPU includes a chamber with a rotor and rotor buckets or mounts to secure containers of an aqueous combination of body tissue and enzyme(s) to be processed. The programming controls spinning of those containers at the selected rotational acceleration speed of the rotor, and for a selected period of time, at the end of which the rotation of the rotor undergoes deceleration and cessation. This operation of the centrifuge is intended to subject the contents of the container to agitation sufficient to cause dissociation of the tissue and preliminary recovery of regenerative cells therefrom. The TPU includes a display and control panel accessible to an operator or user, to enable programming of the various functions of the TPU and the centrifuge therein, as well as to provide feedback to the user regarding the status of the various functions of their operation.

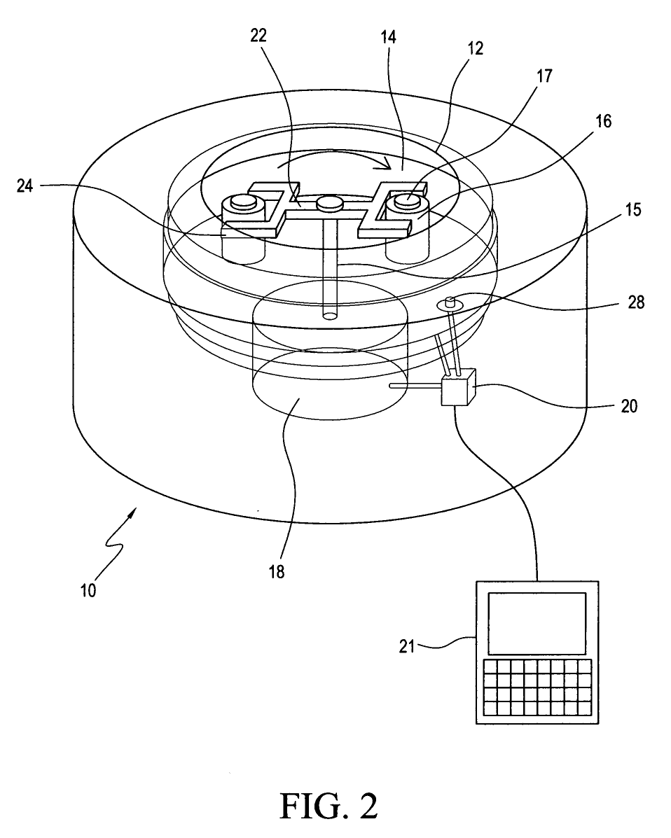

A simplified transparent perspective view of a centrifuge and chamber of a TPU is illustrated in FIG. 2. This is not a preferred embodiment for use with the invention, but merely an illustrative apparatus purely to point to examples of features for the sake of explanation of certain aspects. Referring to the figure, the centrifuge 10 has a wall 12 that creates an inner chamber 14. The diameter of the inner chamber 14 is sufficient to accommodate the size and movement of containers 17 (e.g., processing tube, or wash tube) within rotor buckets such as 16 that are to be subjected to centrifugation in processing the body tissue. Within inner chamber 14 a central axle of rotor 15 is disposed to be driven by a motor 18 to undergo rotation. A programmable controller 20 is connected to a programming console 21 from which an operator may control certain operations. Controller 20 serves to turn the motor 18 on and off according to a programmed regimen (entered by or from console 21) for performing a specific stage or stages of the method of the invention. The controller is also adapted to regulate the temperature within inner chamber 14, by means of an element 28 projecting therein, whereby to similarly regulate the temperature of the contents of each of the containers 17 retained by the respective rotor buckets or mounts 16.

In the example of FIG. 2, each rotor bucket is rotatably suspended between the forked members 24 of respective rotor arms 22. Although only two rotor arms are shown in the figure, a larger number may be provided and utilized. When activated, motor 18 rotates the axle of rotor 15 to spin the arms 22 (clockwise as shown by the curved arrow in this example), and thereby swings the rotatably suspended buckets up at an angle to the rotor axle that depends on the centrifugal force exerted thereon. That is, the buckets are supported from the forked members in such a manner that when the shaft rotates, they swing upward and outward relative to the rotor axle. And in doing so, the gravitational force on each container's contents is directed opposite to the applied centrifugal force thereon. For example, as the centrifugal force is increased, the buckets and the containers retained therein can move from their vertical position shown in FIG. 2 to a position fully 90 degrees upward and outward therefrom. The rate of that movement and the furthest outward position attained by the buckets depends on the speed of rotation of the rotor and the centrifugal force (in g's) applied to the buckets thereby.

Although rotatable buckets or mounts in a swing out mode are coupled to rotor arms in the embodiment of FIG. 2, the same buckets or mounts can be affixed to rotor arms projecting at a fixed upwardly oriented angle from the horizontal from the axle of the rotor, and maintain those buckets or mounts at that fixed angle, as will be described presently for a preferred embodiment.

The temperature of inner chamber 14 is regulated to be within a range from 26 to 42° C., for example, and preferably at or about 37° C., to maintain cell viability during processing of body tissue. The temperature can be regulated by any known method, e.g., closed loop thermal feedback regulation. To keep the temperature constant, the element 28 may constitute a temperature probe operably linked to controller 20, for sensing and maintaining the selected temperature via the controller.

Controller 20 is programmed from the programming console 21 to control starting and stopping of the motor 18, and thereby the ramping up of rotational acceleration of the rotor 15 and the container(s) 17 coupled thereto by the rotor arm(s) 22 and mount(s) 16, and ramping down the rotation (deceleration) according to the interval of stopping the motor, as well as to regulate and maintain the temperature of inner chamber 14. For example, controller programming may be set to rotationally accelerate container 17 to a centrifugal force of between 50 and 4,000 g's, and to maintain that force for a brief interval of 5 to 20 seconds, followed by turning off the motor to decelerate the container to a centrifugal force of 0 g within an interval of 3 seconds. And the controller may be programmed to provide repetitive cycles of the rotational acceleration and deceleration over a period within a range of from 5 to 180 minutes, for example, for agitation of the contents of the containers sufficiently to achieve the desired dissociation of the body tissue therein.

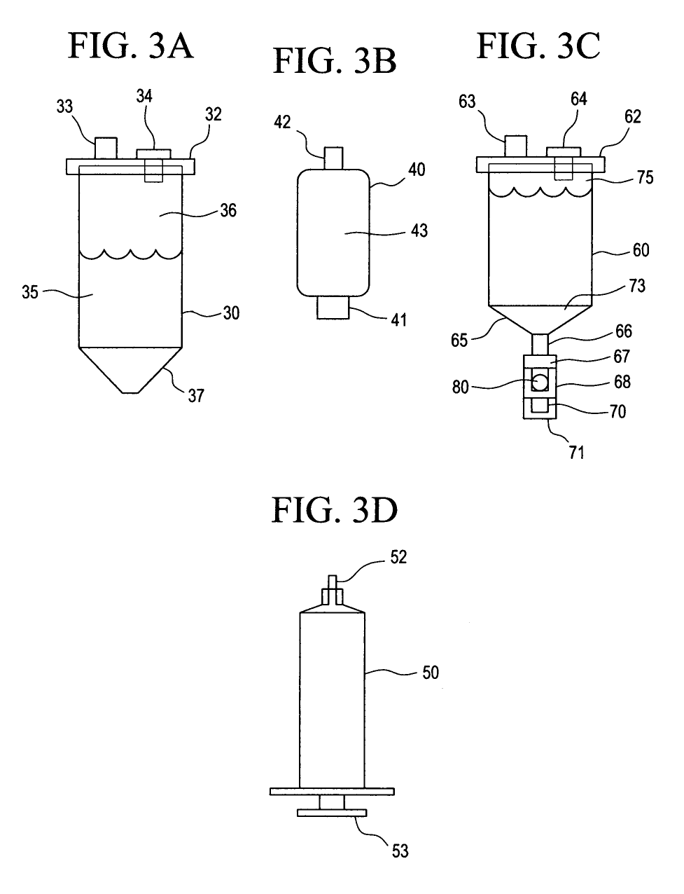

Before further description of the presently preferred overall apparatus and method of the invention, attention is invited to FIG. 3, which illustrates side views of examples of disposable items used in the apparatus, in parts 3A, 3B, 3C and 3D of the figure. These items constitute a set of inexpensive closed sterile disposables for processing of tissue to isolate regenerative cells including stem and other reparative cells. This set of components is utilized with the TPU to dissociate and isolate cells, specifically a regenerative cell mixture from body tissue, and to concentrate those isolated and recovered cells for subsequent use by an attending surgeon or physician in an applicable respective medical therapeutic or diagnostic procedure.

The closed sterile disposable components comprise three assemblies (depicted in stylized form), including a processing tube of FIG. 3A, a particle filter of FIG. 3B, and a wash tube of FIG. 3C. In the illustrated exemplary embodiment, processing tube 30 may be a substantially cylindrical container with a flat cylinder end closed by a cap 32, designated as the top end, having one or more ports or openings 33, 34. The processing tube may be clear or translucent to aid in determining the amount of material in the tube, the status of the processing, and the state of the material within the tube. The processing tube 30 includes a first closable port for ingress of the body tissue and substances used to aid dissociation and egress of dissociated tissue, a second opening for pressure equilibration through a sterile filter for closing the second port. In the embodiment shown, the flat cylinder end contains these two openings, one of which is a female tapered luer connection 34, normally closed by a cap, and the other of which is closed as a filtered sterile vent 33 for pressure equalization. As an aside, the luer connections described throughout this specification are standard tapered male or female (as cited) connections typically used in medical fluid lines, syringes, and needles, and are available in variations of “locking” or “slip tip,” the latter being non-locking, according to their intended use.

The processing tube end 37 distal from the flat cylinder end may be of conical, rounded, flat or other shape. In the illustrated embodiment, the end 37 is shown to be a preferred conical shape to conform to the shape of the receptacle (e.g., the rotor bucket or mount) in which it is to be placed during certain steps of the processing.

When processing is to commence, the processing tube 30 is partially filled with an aqueous combination 35 of body tissue, preferably adipose or lipoaspirate, Ringer's solution, and proteolytic enzyme (e.g., one or more collagenases such as type I and/or type II collagenases, and a neutral protease such as thermolysin, trypsin, or mixtures thereof), to leave a void or open air space 36 that allows the aqueous combination 35 to undergo sloshing when its container 30 is subjected to agitation. To that end, the processing tube is placed securely in a rotor bucket or mount (having the preferred conical shape at its base) on arms attached to the rotor of the centrifuge, where the contents of the tube are to undergo processing by application of heat and agitation (by two or more cycles of rotational acceleration and deceleration thereof) to promote dissociation of the tissue toward isolation of its cellular structure, principally its regenerative cells. As an aid to enzymatic dissociation, but in order also to maintain cell viability, the chamber is heated in a controlled manner, and maintained at a temperature of approximately 37° C.

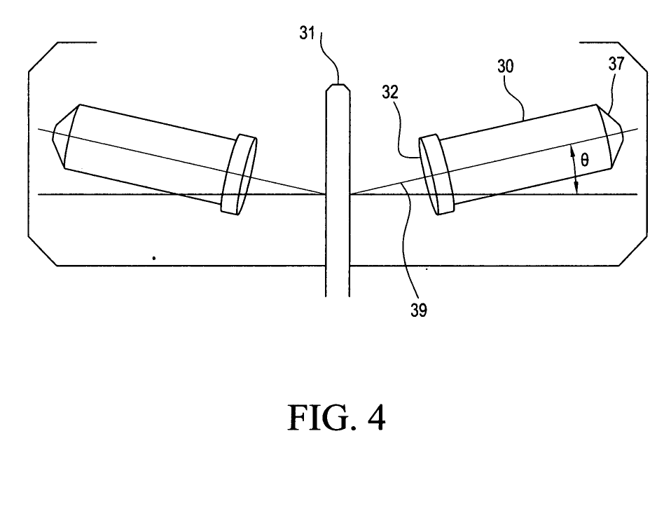

In an illustrative exemplary embodiment, and with reference now to FIG. 4, the processing tube 30 (first container) of FIG. 3A is secured within a rotor arm mount or bucket (not shown in this figure, for the sake of simplicity) with its conical end 37 projecting from the mount away from the rotor 31 central axis, and angled upwardly at an angle θ by its rotor arm (depicted only as a central axis 39 of the arm) so that the conical end 37 of the processing tube is elevated relative to its flat cylinder end 32. In this embodiment, the angle of tube deployment is set at a fixed angle θ (e.g., an angle ranging from 1 degree to less than 90 degrees). For example, the angle θ may be fixed at 12 degrees, and the container or tube 30 may be coupled to rotor arm 39 with the arm in an orientation inverted from its typical orientation on a centrifuge rotor.

With this positioning, when the processing tube is at or brought to rest, aqueous material therein moves under the force of gravity along the tube toward the end 32 proximal to the rotor 31 central axis, and away from the tube's conical end 37 distal to the rotor's central axis. And when the apparatus undergoes centrifugation, the centrifugal force on the processing tube causes its contents to move away from the rotor axis and toward the conical end of the tube to fill any void air space left in the container. Cessation of rotation of the rotor by switching off the motor has the opposite effect, and cycles of spinning and stopping thus have the effect of considerable agitation of the processing tube contents. The agitation is cycled at least twice, but may be repeated in many more cycles of repetitive rotational acceleration and deceleration. In any event, the extent of the agitation is made sufficient that the contents of the processing tube tend to undergo dissociation into cells, primarily regenerative owing to the rich vascularization of adipose tissue, and spent (i.e., any non-dissociated) tissue pieces and enzyme. The agitation and all recovery thereby depends on various factors including the centrifugal forces applied, the shape and size of the container, the ratio and amount of filling to air space in the container, the viscosity and composition of the filling, the speed of acceleration and deceleration and the number of cycles thereof.

Returning to the components shown in FIG. 3, a particle filter 40 (FIG. 3B), a unit of preferably flat configuration with spaced apart sides, has a male luer port 42 on one end, and a female luer port 41 on the other end. In use for further processing the dissociated body tissue, the male luer connector 42 of the filter 40 is coupled to the female luer port 34 of the processing tube 30, and the open tip 52 of a syringe 50 (FIG. 3D) is coupled to the female luer port 41 of filter 40. The plunger of the syringe is movable outward and inward of the syringe body or barrel by the typical operation of withdrawal and re-insertion of the syringe handle 53. Attachment in this manner allows the now dissociated contents of the processing tube 30 to be vacuum-extracted from the processing tube upon withdrawing the syringe plunger, thereby transferring the contents of the processing tube through the filter 40. An intervening mesh 43 is disposed within the tubular filter 40 between its two ends, so the contents from the processing tube are filtered as they are drawn therethrough. Mesh 43 has its openings sized in a range from 40 μm to 300 μm, preferably 100 μm, for example, to allow passage of cells of interest, primarily regenerative cells, through the mesh and out of the female luer port 41 of filter 40, but to trap the larger portions of tissue that have not undergone complete dissociation (i.e., are non-dissociated). Consequently, the resulting mixture of cells and enzyme exiting the tubular particle filter 40 is drawn into the syringe 50.

The cell mixture must then be extracted from the processed tissue and matrix. This is accomplished, according to the invention, by introducing the filtered cell/enzyme mixture into a wash tube 60 (second container, FIG. 3C). The syringe 50 containing the dissociated cell/enzyme mixture is attached at its open tip 52 to a female luer port 64 at a flat circular capped end 62 of the cylindrical wash tube. The mixture is then introduced into the closed wash tube by depressing the syringe handle 53 to correspondingly force its attached plunger into the barrel of the syringe, and thereby dispense the mixture from syringe tip 52. The wash tube has a 0.2 μm sterile filter vent 63 on its flat circular end 62 for pressure equalization to allow displaced air within the wash tube to escape as the wash tube is filled or to allow air to flow in as the wash tube is emptied.

In the presently preferred embodiment illustrated in FIG. 3C, the wash tube 60 is cylindrical in shape, with a conical taper 65 at its end opposite its capped flat circular end 62. The taper itself ends in a female open appendage or port 66 to a smaller diameter open cylinder 67, and thence into a reservoir 68, which is sealed at its end 70. The aqueous mixture 73 now in the wash tube occupies the majority of the tube's volume and leaves only a small empty space 75 on top. The mixture 73 includes cells, enzymatic reagent, excess liquid, and other material with specific gravity less than 1.0 that can be separated from the cellular content by gravitational force.

This removal is accomplished by first securing the wash tube containing mixture 73 in a rotor bucket or mount of the centrifuge of the TPU, in a manner corresponding to that described in the example of the processing tube earlier herein, with reference to FIG. 4. The wash tube is positioned in its swing out bucket rotor mount with its conical and reservoir end 70 distal from the rotor axis and its flat end 62 proximal to the rotor axis when undergoing centrifugation. Accordingly, the wash tube is subjected to centrifugal force to induce sedimentation of the cells at the conical tapered end 65 of the wash tube and port 66, away from the remainder of the mixture.

As a result, a pellet 80 of cells derived from the original body tissue is formed by sedimentation and moves along the smaller cylinder 67 and into the reservoir 68. The reservoir end 70 is preferably sealed by a flexible material such as a silicone cup with a slit (not shown) that acts as a valve but allows passage of the cell mixture pellet 80 when opened; however assuring retention of the cell mixture pellet when undisturbed. The pellet may then be removed by inserting a male luer connection through the slit, or by application of a second syringe tip through the slit for its withdrawal. Alternatively, the reservoir may be sealed by a swabable luer connector that is normally held closed by a cap 71, but which can be opened by application of a syringe with its typical male luer connection thereto.

The recovered pellet, which incorporates a substantial population of regenerative cells, is then available for a continued washing process or for use in any of the medical procedures mentioned above, and any others for which it may be advantageous.

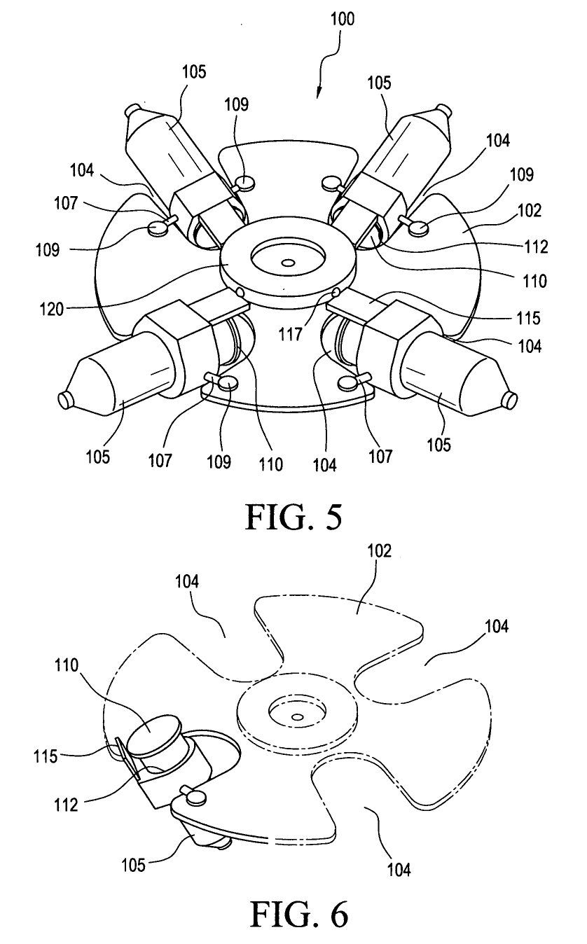

In addition, and in more detail to FIG. 2, FIGS. 5 and 6 are partial perspective and phantom perspective views of a centrifuge rotor with buckets or mounts pivotally fastened thereto for fixed orientation and pivotally-determined orientation, respectively. The figures are of the same basic configuration and both are suitable for use in centrifugation of either a first closed sterile container (e.g., the processing tube) or a second closed sterile container (e.g., the wash tube). Referring to FIG. 5, the rotor 100 is essentially a circular flat metal plate 102 of suitable thickness with plural cutaway openings 104 for accommodating an equal number of buckets 105 to pivotally fastened to the plate by pins 107 secured to either side of each bucket and allowed to rotate within matching holes of posts 109 riveted or otherwise securely fastened to the plate 102. The pivotal fastenings for the buckets are arranged relative to their respective cutaway openings 104 to allow the buckets to pivot freely from an unrestrained (resting) position shown in FIG. 6 to the position shown in FIG. 5, and beyond. And to do so when a container 110 is seated in the receptacle 112 of a bucket and secured therein in preparation for centrifugation.

In the exemplary illustration of FIG. 5, each of the buckets (and its container seated therein) is restrained in an orientation of its axis at an angle θ to the surface of the plate 102 by a lever arm 115 of the bucket when the end of the arm is secured beneath a respective projection 117 on the central fitting 120 that secures the plate 102 to the rotor axle (not shown). The restrained orientation at angle θ is preferred for the processing tube in its repetitive cycling of rotational acceleration and deceleration, although the unrestrained freely pivotal orientation may be used if desired. In the case of subjecting the wash tube to a standard centrifugation run for sedimentation of cells from the aqueous mixture, the unrestrained pivotal orientation is preferred.

Operation of either of the two configurations for centrifugation is as discussed earlier herein.

It will be understood that the apparatus illustrated in FIGS. 5-6 is not to scale or proportion and that certain features may be exaggerated or distorted for illustrative purposes. And the same comment applies to the depictions in the other figures of the drawings.

The system or apparatus employed during processing of the body tissue constitutes a closed system, which may include, without limitation, mechanisms such as sterile filters, luer connectors and tubings between containers, filters and syringes. Also, connection of the containers and filter by fixed tubings, and respective mechanical forces applied to move and transfer contents from one site to another, are part of the invention. A closed system method of transfer prevents the various materials undergoing transfer between one location and another from being contaminated by pathogens external to the tissue sample and/or the tissue or other material containers, such as but not limited to bacteria, viruses, and the like from entering into the containers or other receptacles. The closed system method reduces the necessity to perform additional steps prior to administration of the regenerative cells into a recipient subject, which may be the original donor. As used herein, including the claims, the numerical notation of ‘first container’ and ‘second container’ denotes the order of usage of the containers. The containers may be of the same type or of different type, but in the embodiment and method of interest herein, are what are characterized as processing tube and wash tube respectively.

The invention will be further described with respect to the following Example which is not meant to limit the invention, but rather to further exemplify the various methods and embodiments of processing apparatus.

### Example

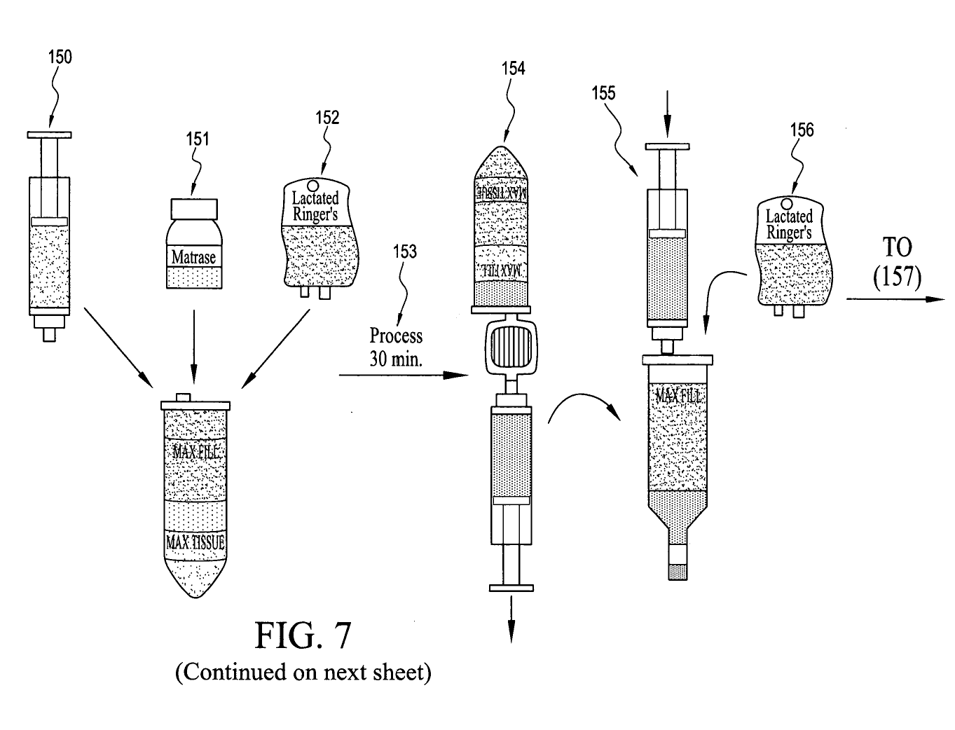

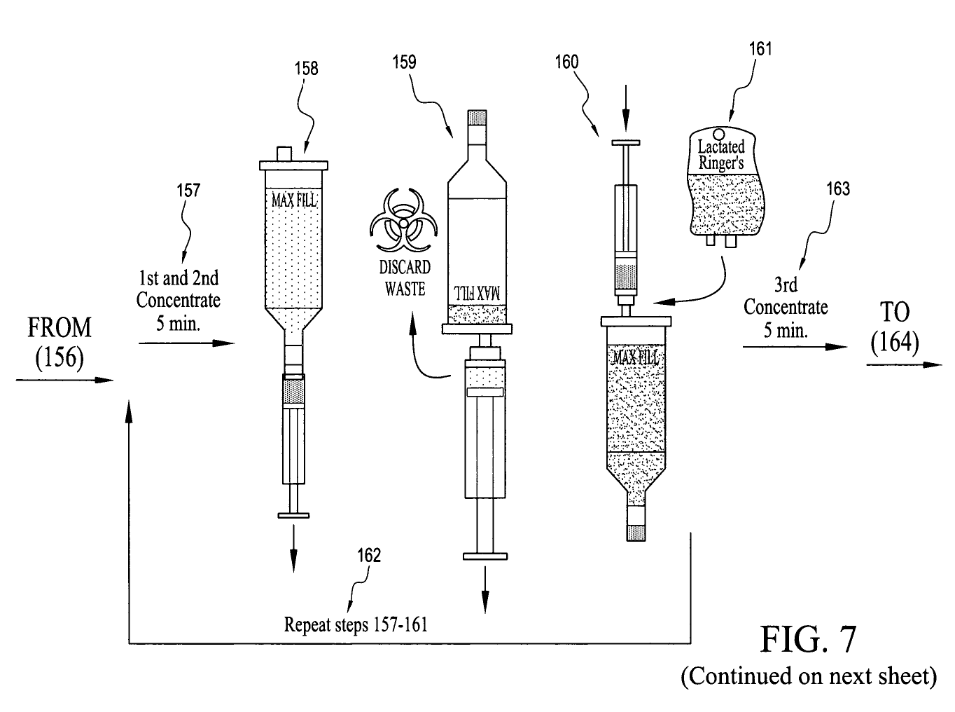

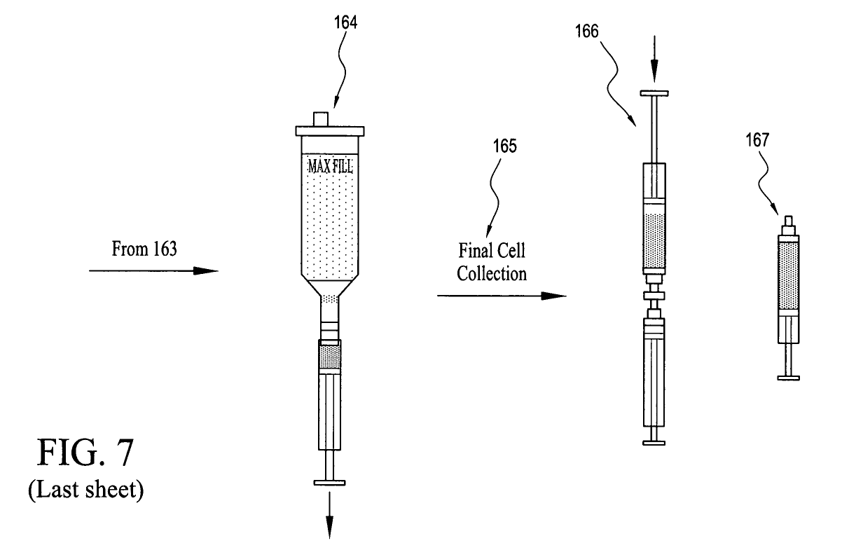

FIG. 7 constitutes an exemplary closed tissue processing system apparatus and flow diagram of an exemplary closed system method of the invention. It will be observed that FIG. 7 encompasses multiple sheets of the drawings, with multiple stages or steps (designated for convenience by reference numbers 150-167), including several that have subparts, which serve to more clearly and fully describe and explain the various operations entailed by the system and method. And it is to be understood that although the various stages may depict only one tube (container) as being used or undergoing processing, multiple containers may be used or processed in the same manner for each stage in the same time frame(s).

In step 150, a sample of adipose tissue obtained from a donor subject is loaded in a processing tube (first sterile closed container) up to but not exceeding a line labeled MAX TISSUE. In the presently preferred embodiment, the processing tube will hold approximately 24 mL of tissue when filled to the MAX TISSUE line. Then, 2.5 mL of reconstituted MATRASE™ (trademark of InGeneron Incorporated of Houston, Tex., for its mixture of collagenase and dispase enzymes) is added to the processing tube from a 10 mL syringe (step 151). Finally, in step 152, lactated Ringer's (Ringer's lactate solution, readily available from supply houses, essentially a saline solution with some added Ca and Mg, at times referred to herein simply as “Ringer's”) is preheated to approximately body temperature, generally specified as approximately 37° C., but preferably not more than 40° C. and added to the processing tube up to the MAX FILL line (44 mL, for the processing tube) from a 60 mL lactated Ringer's syringe.

The contents of the processing tube are then processed in the TPU, which is a modified centrifuge. The device is programmed to spin up, slow down, and stop approximately 6 times per minute. The speed top reached in each spin is approximately 15 RCF, although that number is not critical. The speed should be sufficient to move the tissue to the distal end (conical end, in this embodiment) of the tube relative to the rotor central axis, against the force of gravity, and will depend on the angle θ (FIG. 4), over the selected time interval for processing, to produce dissociation of the tissue. Lipoaspirate, as the body tissue, is processed for 30 minutes (step 153). Minced solid adipose tissue is processed for 60 minutes.

In step 154, the dissociated tissue, cells, reagents and any other substances derived following the 30 minutes of processing are transferred from the processing tube by vacuum-extraction performed by withdrawing the plunger of a 60 mL syringe coupled via its luer connection to the luer connector of the particle filter, which in turn has its luer connection at its opposite end luer-connected to the egress port of the processing tube. Thus, the contents of the processing tube minus any non-dissociated tissue and other large-sized particles trapped within the 100-300 μm mesh of the particle filter are drawn into the syringe. Those filtered contents, consisting primarily of cells and reagent, are then transferred via luer connection from the syringe into the wash tube (the second closed sterile container) in step 155, followed by adding lactated Ringer's up to a MAX FILL line on the wash tube (43 mL). The volume of the filtered contents transferred into the wash tube can be as much as that MAX FILL, but is generally less, so some room remains for adding the Ringer's, using a 60 mL Lactated Ringer's syringe (step 156). The user can “top up” the wash tube to the MAX FILL line with Ringer's, which is the wash fluid.

The wash tube is then placed in a centrifuge for concentration of the cells by sedimentation in one 5 minute spin (step 157). Although the latter spin may be viewed as spin up and spin down, it is performed only once for the wash cycle (referred to herein as a standard run of centrifugation, although it might be performed once again but only if a second complete wash cycle is performed). Multiple start and stop cycles are performed only during processing.

In step 158, 1.5 mL cells are extracted from the reservoir appended to the wash tube, by use of a 3 mL syringe via a luer connection. After the cells have been removed, the remaining liquid in the wash tube is extracted via an egress luer port at the flat end of the wash tube using a 60 mL tissue syringe (the term “tissue” syringe being used here solely to distinguish it from the Ringer's syringe, and may be the same one as was used in step 150 to transfer tissue), and the contents of the tissue syringe are then discharged into a sterile waste container (step 159). The 1.5 mL cells are returned to the wash tube (step 160), by ejection from the 3 mL syringe via a luer connection to the ingress port of the wash tube. Ringer's is added as wash fluid to the MAX FILL line of the wash tube from a 60 mL Lactated Ringer's syringe (step 161) via the same luer connection, after the cells have been dispensed into the wash tube and the 3 mL dispensing syringe removed.

Preferably, but optionally, a step 162 is performed as a one-time repeat of the concentrate step and of steps 157-161, using the same syringes as had been used in the initial performance of steps 157-161. So two wash steps or stages are performed, each of which includes a concentration step (spin, by a 5 minute standard centrifugation run) and wash.

A third spin is performed on the remaining cell mixture/enzyme mixture/Ringer's contents of the wash tube after the one-time-repeated (if used) wash (step 162), again as a standard centrifugation run for a period of 5 minutes (step 163) in this example, for further cell concentration and preliminary collection. At the conclusion of that third spin, pelletized cells are extracted from the reservoir appended to the wash tube (step 164), which may be performed using the 3 mL syringe from step 158. The third spin is the last pull (step 165), and represents an effort to extract as many cells as possible from the mixture in the wash tube at the conclusion of step 164. This results in a larger cell pellet than had been previously retrieved from the reservoir; as much as 3 mL.

In the final cell collection (step 165), the pelletized cells are discharged from the barrel of the 3 mL syringe used for extracting them in step 164, by depressing the plunger of the syringe after coupling it through luer connection to a new 3 mL syringe (step 166). The cells are pushed through the connection and into the barrel of the new syringe to remove clumps. The pellet into which the cells have been formed represents the original clump, and the desire here is to break it up. The first passage through the luer connection of the two syringes typically serves that purpose, but there are or may be bits consisting of cells still hanging together. These are dispersed by pushing the cells back and forth between the two syringes (within step 166) to finally place the dispersed cells in the new syringe (step 167).

The retained population of cells in the new syringe is then available for use at the discretion of the attending physician or surgeon, which may be in a current medical procedure, or the cells may be banked, as by cryopreservation, for later use.

Where the cells to be isolated and recovered are regenerative cells, that designation being used herein in its broadest sense, the body tissue to be processed is preferably adipose tissue or lipoaspirate, or other tissue rich in cells.

Thus, the invention may be characterized in one of its aspects as apparatus for isolation and recovery of cells from body tissue of a human or animal subject, including at least a first closed sterile container for dissociation of the tissue by subjection therein to a reagent that promotes tissue dissociation; a programmable centrifuge for receiving and subjecting the first container to repetitive cycles of rotational acceleration and deceleration to further facilitate dissociation; a particle filter for passage of cells released by tissue dissociation in the first container while trapping larger bits of non-dissociated tissue and other materials therefrom; a second closed sterile container for receiving contents discharged from the filter comprising cells, connective material, and dissociation reagent, along with liquid added for washing thereof; the centrifuge for receiving and subjecting the second container to centrifugation for isolating cells from washed contents therein and concentration of the isolated cells for recovery from the second container.

The centrifuge is programmed to subject the first container to at least two cycles of rotational acceleration and deceleration, with the second cycle substantially immediately following cessation of the first cycle. The first container has at least one closable port for ingress of tissue and dissociation reagent and egress of contents derived from dissociation in said first container, and at least one port closed by a pressure equilibration filter, which includes a filter membrane sized to allow air, but not liquid or particles such as bacteria, to flow through the membrane, and each of the ports is a tapered luer connection. The particle filter is a unit having an inlet port and an outlet port, and houses a mesh between those ports to filter material entering the inlet port and allow that portion of the entering material that passes through the entire mesh to exit the outlet port. Openings in the mesh range from 100 μm to 300 μm.

The second container is cylindrical with a flat end having at least one first closable port for ingress of contents discharged from the particle filter, along with wash liquid, and at least one filter port closed by a pressure equilibration filter of the same type as in the first container, and at least one second closable port for egress of isolated cells to be recovered. The opposite end of the second container is a tapered cone in which the taper commencing closer to the conical end than the flat end, and the conical end opens into a smaller cylinder having a reservoir at its end for collection of isolated cells in pellet form. The first and second closable ports of said second container are tapered luer ports, corresponding to ports in the first container. The isolated cells in the second container are preferably transferred back as input thereto, along with liquid added for a second washing, and undergo another run of centrifugation for further concentration into the bunched cell pellet form. The reservoir has a flexible component or membrane for retaining the isolated cells in pellet form therein, and a slit in the flexible component for retrieval of the isolated cells pellet from the reservoir. Whether a slit or a membrane is used, either one is normally closed but opened by the tip of a syringe to retrieve the cell pellet.

In another of its aspects, the invention may be characterized as a method for isolation and recovery of cells from body tissue of a human or animal subject. Steps of the method include introducing the tissue and a dissociation reagent into the first closed sterile container for initial dissociation of the tissue; subjecting the first container to repetitive cycles of rotational acceleration and deceleration in a centrifuge to further facilitate tissue dissociation; filtering the contents of the first container derived from the dissociation of tissue therein for passage of cells released by dissociated tissue while trapping larger bits of non-dissociated tissue and other materials therefrom; and discharging contents that have passed entirely through the filter, including cells, connective material, and dissociation reagent, into the second closed sterile container, along with wash liquid added for washing thereof. A syringe is used with its luer end coupled by sterile connection to the outlet port of the filtering unit for vacuum-extraction to transfer contents of the first container derived from the dissociation of tissue therein into the inlet port of the filtering unit for passage through the filter, whereby to capture the cells and any other material that exits that outlet port in the syringe for transfer to the second container. The method continues with subjecting the second container to a run of centrifugation for isolating cells from washed contents, and concentration of the isolated cells for recovery of a bunched cell pellet from the reservoir at the conical end of the second container. The volume of fluid removed that contains the concentrated cells in the pellet is less than 10% of the initial filling volume of the container.

The method includes programming the centrifuge to subject the first container, and thereby its contents, to at least two cycles of rotational acceleration and deceleration, with the second cycle substantially immediately following cessation of the first cycle. And ultimately transferring isolated cells in the second container back as input thereto for the second wash, and a second run of centrifugation of that container. The method further includes limiting the volume of tissue and dissociation agent introduced, to leave air space at the top of the first container, and placing the first container in the centrifuge in an angled orientation with an end of the first container distal from the centrifuge axis of rotation at a level higher than the opposite end of the first container proximal to the axis of rotation so that centrifugal force moves the combination of tissue and dissociation reagent upward and outward from the proximal end toward the distal end during the portion of each cycle of acceleration, and gravitational force moves the combination downward and inward from the distal end toward the proximal end during the portion of each cycle of deceleration and cessation of centrifugation, when the first container is undergoing its repetitive rotational acceleration and deceleration.

Although a presently preferred embodiment and method of the invention have been described in this specification, it should be understood that various alternatives may be suggested or derived from the disclosure to persons skilled in the relevant arts, without departing from the spirit and scope of the invention. Accordingly, the invention is intended to be construed and interpreted from the appended claims in determining the protection to be afforded to the applicant herein.

1. Apparatus for recovering regenerative cells from body tissue, comprising a first container to receive a sample of the body tissue and a proteolytic enzyme; a centrifuge adapted for subjecting the contents of the container to at least one cycle of rotational acceleration and deceleration for dissociation of the tissue; a filter to pass the regenerative cells while retaining tissue too large to pass through the filter; a syringe for extracting the contents of the first container through the filter into a second container for then subjecting its contents to centrifugation and thereby to separate the regenerative cells from unwanted substances of lower specific gravity than that of the regenerative cells; and a reservoir integral to the bottom of the second container for recovering the regenerative cells through an additional opening at the bottom of the second container.

2. The apparatus of claim 1 wherein the opening at the bottom of the second container comprises a valve.

3. The apparatus of claim 2 wherein said valve is a swabable luer.

4. The apparatus of claim 1, wherein the proteolytic enzyme comprises a mixture of one or more of a protease and collagenase.

5. The apparatus of claim 1, including a temperature-controlled heater for maintaining the temperature of the centrifuge interior, and thereby, of the contents of a container received therein, in a predetermined range of from about 32° C. to about 42° C. during the agitation thereof.

6. The apparatus of claim 1, wherein the centrifuge includes a central rotor that enables a first configuration to receive said first container in a fixed position, wherein the end of said first container closest to the central rotor is lower than the other end, and where said central rotor enables an alternative second configuration for said second container to swing free in a standard swing out mode.

7. A method for cell recovery from tissue, comprising inserting the tissue into a first closed internally sterile container along with a dissociation reagent through a single connective opening at the top of the container, while providing equilibration of pressure through a filtered vent integral to said first container; agitating the resulting mixture of tissue and reagent to enhance dissociation of cells from the tissue in said first container; transferring the processed content of said first container through a particle filter into a second closed internally sterile container, adding wash fluid thereto and separating the cells from unwanted material by centrifugal force-induced sedimentation to produce a cell pellet at the bottom of said second container; and recovering said cell pellet through an opening at the bottom of the second container.

8. The method of claim 7, including repetitively transferring the removed cell pellet with a syringe into the second container together with clean wash fluid, and initiating additional centrifugation of the second container to produce sedimentation and further concentration of cells for separation and cleansing of the pellet from debris and residual enzyme.

9. The method of claim 7, including partially filling the first container with tissue and reagent as its contents, that preserves sufficient air space within the partially filled first container to enable enhanced movement of said contents when subjected to agitation.

10. The method of claim 9, including subjecting said first container to centrifugation in the form of repetitive cycles of rotational acceleration and deceleration, as said agitation of the contents of said first container.

11. A system for isolation and recovery of cells from body tissue, comprising a first container having a port on one end to receive said tissue for dissociation thereof to at least partially isolate and recover cells therefrom, a particle filter arranged for receiving cells and nondissociated tissue from the first container to further isolate and recover cells by allowing passage of the cells therethrough while trapping nondissociated tissue therein, and a second container having a port adapted to receive contents of the filter passed therethrough for separation of the isolated and recovered cells from unwanted material in said contents through application of centrifugal force in combination with gravitational force to the second container, the second container having a second port on its bottom relative to direction of gravitational force for removal of separated cells collected thereon as a result of sedimentation in response to said centrifugal force.

12. The system of claim 11, wherein said port of said first container is adapted to be open for ingress and egress of tissue and substances used to facilitate dissociation of tissue, and closable during dissociation of the tissue, and the first-named port of said second container is open for ingress of said contents of the filter and closable during said application of centrifugal force, and each of said first and second containers has an integral pressure equilibration filter.

13. The system of claim 12, wherein each said pressure equilibration filter comprises a filter membrane sized to allow air, but not liquid or particles such as bacteria, to flow therethrough.

14. The system of claim 12 wherein the closable ports of said first and second containers are tapered luer ports.

15. The system of claim 12, wherein said adaptation of the port of the first container and said arrangement of the particle filter comprise connection of the first container and the particle filter by tubings and luer connections, and movement of contents of the first container and transfer thereof to and through the particle filter are induced by application of respective mechanical forces thereto.

16. A method to separate a cell population of interest from processed body tissue, comprising:
transferring the processed tissue into a container together with fluid,
subjecting the container and its contents to centrifugal force for concentration of the cells from the fluid,
forcing the concentrated cells to undergo sedimentation at the bottom of the container, and
removing the cells of interest through an opening at said bottom of the container,
wherein the volume of the fluid removed that contains the concentrated cells is less than 10% of the initial filling volume of the container.

17. An apparatus to separate a cell population of interest from processed tissue comprising:
a container to receive processed tissue and fluid,
said container adapted to be subjected to centrifugal force to cause cells to undergo concentration and sedimentation at the bottom of said container,
said container having an opening at the bottom for removal of concentrated cells of interest,
wherein the container is shaped with an integral reservoir at the bottom of the container for retention of the concentrated cells in the form of a cell pellet.

18. The apparatus of claim 17 wherein the container is equipped with openings at both of its opposite ends.

19. The apparatus of claim 17 wherein the reservoir is closed by a valve.

20. The apparatus of claim 19 wherein said valve is a swabable luer connector.
